(19) United States
(12) Patent Application Publication
(10) Pub. No.: US 2022/0347354 A1
(43) Pub. Date: Nov. 3, 2022

(54) URINARY CATHETERS AND METHODS FOR PREVENTING BACTERIAL INFECTIONS

(71) Applicant: Hollister Incorporated, Libertyville, IL (US)

(72) Inventors: Tadas S. Sileika, Lake Forest, IL (US); Nicole L. Dean, Antioch, IL (US); Jacqueline MJ Pettigrew, Lindenhurst, IL (US)

(21) Appl. No.: 17/771,126

(22) PCT Filed: Oct. 27, 2020
(86) PCT No.: PCT/US20/57488

§ 371 Date: Apr. 22, 2022

Related U.S. Application Data
(60) Provisional application No. 62/926,891, filed on Oct. 28, 2019.

(51) Int. Cl. A61L 29/08; A61L 29/04; A61L 29/16

(52) U.S. Cl. CPC A61L 29/08; A61L 29/046; A61L 29/043; A61L 29/16

(57) ABSTRACT

Urinary catheters and methods for preventing bacterial infections.

The present application claims the benefit of and prior to U.S. Patent Application No. 62/926,891, filed Oct. 28, 2019, which is hereby incorporated herein by reference.

### TECHNICAL FIELD

The present disclosure generally relates to urinary catheters and methods for preventing bacterial infections, and more particularly, to catheters and methods for preventing urinary tract infections. The present disclosure also relates to urinary catheters and methods that inhibit the motility of bacteria within the urethra.

### BACKGROUND

It is desirable for urinary catheters to have a lubricated or lubricious outer surface to increase comfort of the patient during insertion into and/or removal from the body. One method for rendering the surface of a urinary catheter lubricious is to coat at least the insertable portion of the catheter with a lubricious hydrophilic coating. Another method is to apply a lubricant to the urinary catheter. Lubricity of the catheter minimizes soft tissue damage and reduces overall discomfort during use of the medical device.

Although catheters are typically prepared in sterile environments and are provided with safe handling instructions, catheter users are at risk of contracting a urinary tract infection due to several different factors. For example, the catheter may become contaminated during use and introduce bacteria into the urethra. The catheter also may carrier bacteria along the urinary tract. Additionally, because urine is voided from the bladder through the catheter, urine does not flow directly through the urethra. Thus, urine is no longer a factor in flushing out the urethra or wetting of the urethral tissue.

Therefore, there is a need for catheters, which reduce the risk of urinary tract infections.

### SUMMARY

There are several aspects of the present subject matter, which may be embodied separately or together in the devices and systems described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In one aspect, a urinary catheter product for insertion into the urethra, wherein the urethra contains urethral mucus having a viscosity. The catheter product includes, a catheter shaft, and a compound for retaining and/or increasing the viscosity of the urethral mucus.

### BRIEF DESCRIPTION OF THE DRAWINGS

FIG. 1 is a plan view of one embodiment of the present disclosure; and

FIG. 2 is a plan view of one embodiment of a catheter of the present disclosure.

### DETAILED DESCRIPTION OF THE ILLUSTRATED EMBODIMENTS

The embodiments disclosed herein are for the purpose of providing a description of the present subject matter, and it is understood that the subject matter may be embodied in various other forms and combinations not shown in detail. Therefore, specific embodiments and features disclosed herein are not to be interpreted as limiting the subject matter as defined in the accompanying claims.

For catheter users, whether in-dwelling or intermittent, urine is no longer a factor that participates in regular flushing or wetting of the urethra. Because the users are unable to void directly through the urethra, the urethra no longer experiences the continuous flushing and re-wetting of the tissues that normally occurs during active voluntary urination. When the mechanism fails, the risk of urinary tract infections increases.

Urinary tract infections can be a result of bacteria swarming and traveling up through the urethra towards the bladder. Traveling of bacteria up the urethra is typically not a concern for those who can perform voluntary urination, because normal voiding tends to wash out and eliminate the bacteria from urethra, thereby prohibiting it from traveling toward the bladder and causing infection.

There are certain species of bacteria that are considered good “swimmers,” which swarm and move when they receive certain signals from the surrounding environment. These bacteria are capable of movement even on solid surfaces through excretion of their own extracellular fluids. These motile bacteria also are known to become more pathogenic and increase their motility organelles, such as flagella (a process called hyperflagellation), when sensing certain signals, such as decreased viscosity in the fluids and mucus in their surrounding environment.

In a non-voiding patient, the urethral environment will be fairly viscous, due to the presence of mucosal secretions. When a hydrophilic catheter or a catheter lubricated with a water-based gel is introduced into the urethra, such introduction will likely deposit additional moisture, diluting the secretions, thus decreasing the overall viscosity of the urethral mucus and the surrounding urethral environment. This decrease in the urethral mucus viscosity provides a signal that activates the motility of the bacteria.

The catheter products disclosed herein inhibit bacteria mobility and/or mitigate the risk of bacteria swarming and traveling proximally up the urethra toward the bladder. The catheter products include a compound for retaining and/or increasing the viscosity of the urethral mucus (which includes the fluids and other substances within the urethra). As discussed above, when the viscosity of the urethral mucus is decreased, such decrease can provide a signal to the bacteria that activates motility of bacteria. When the viscosity of the urethral mucus is retained and/or increased, there is no decreased viscosity signal and the motility of bacteria is not activated. Thus, the bacteria remain static and are not activated to swarm and travel up the urethra toward the bladder.

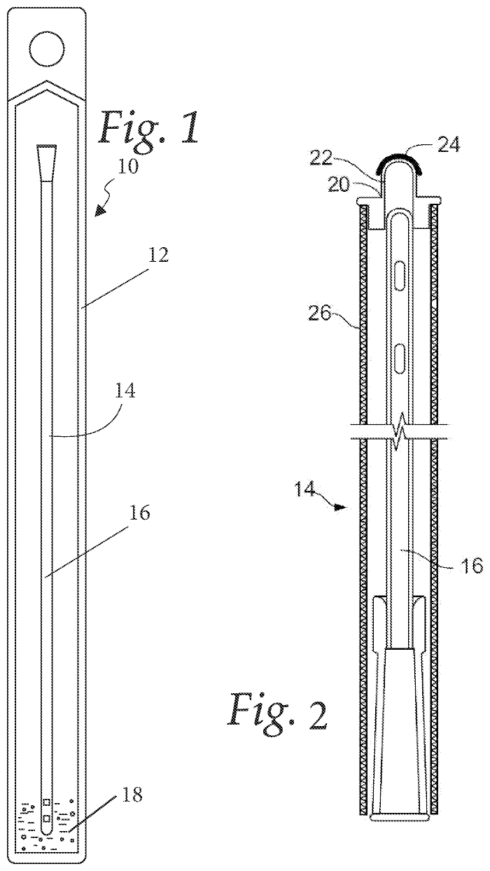

The compound for retaining and/or increasing the viscosity of the urethral mucous may be included in any component of a catheter product. For example, referring to FIG. 1, a catheter product 10 includes a package 12 including a catheter 14 having a catheter shaft 16. The outer surface of the catheter shaft 16 may be hydrophilic. For example, the catheter 16 may be coated with a hydrophilic coating. The hydrophilic surface of the catheter shaft 16 becomes lubricious when wetted with a hydration medium 18 (water or saline). In one alternative, the compound for retaining and/or increasing the viscosity of the urethral mucous may be contained in the hydrophilic material (e.g., the hydrophilic coating) and/or the hydration medium. When the catheter product includes a lubricating gel, the compound may be included in the lubricating gel.

Referring to FIG. 2, when the catheter product includes an introducer aid 20 that may be inserted into the urethral opening and which the catheter is inserted through, the introducer aid 20 or the tip 22 thereof may include the compound 24. When the catheter shaft 16 is inserted through the introducer aid 20, the retaining and/or viscosity increasing compound may be disposed onto the catheter shaft 16 wherein the catheter distributes the compound along the urethra. Additionally, a sleeve 26 may be attached to the introducer aid, wherein the sleeve surrounds the catheter.

In one embodiment, the catheter includes a compound that dries out the urethral environment and/or the urethral mucus, by removing water from the urethral environment. The removal of water from the urethral environment maintains and/or decreases the viscosity of the environment. In one example, the compound removes any residual moisture following catheterization. The residual moisture could be associated with either the presence of mucosal secretions and/or the water left behind from the insertion of hydrophilic catheter or water based gel-lubricant catheter.

The compound included in the catheter product to assist in drying out the urethral environment may be a desiccant that removes moisture from the urethral mucus. The desiccant may include one or more of alcohol, volatile solvents, antihistamines, and loperamide hydrochloride. As mentioned above, the desiccant may be included in a hydrophilic coating, hydration medium, gel-lubricant or insertion tip of the catheter.

In another embodiment, the compound that maintains or increases the viscosity of the urethral environment may be a thickening agent that the catheter deposits into the urethral environment. The thickening agent may offset any moisture that is placed into the urethral environment by catheterization and/or the thickening agents may increase or maintain the original viscosity of the urethral environment. When the viscosity of the urethral mucus is retained and/or increased, there is no decreased viscosity signal and the motility of bacteria is not activated. Thus, the bacteria remain static and are not activated to swarm and travel up the urethra toward the bladder.

The thickening agent could be for example, gelling agents, such as agar or gelatin, or physical hydrogels, such as that formed by polysaccharides (e.g., chitosan, xanthan gum or hyaluronic acid).

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

What is claimed is:

1. A urinary catheter product for insertion into a urethra, wherein the urethra contains urethral mucus having a viscosity, the catheter product comprising:
a catheter shaft; and
a compound for retaining and/or increasing the viscosity of the urethral mucus.

2. The urinary catheter product of claim 1, further including a hydrophilic coating on the catheter shaft, where the compound is contained in the hydrophilic coating.

3. The urinary catheter product of claim 1, further including a hydrophilic coating on the catheter shaft and a hydration medium for hydrating the hydrophilic coating, wherein the compound is contained in the hydration medium.

4. The urinary catheter product of claim 1, further including a lubricant for lubricating the catheter shaft, wherein the lubricant includes the compound.

5. The urinary catheter product of claim 1, wherein the compound comprises a desiccant that removes moisture from the urethral mucus.

6. The urinary catheter product of claim 5, wherein the desiccant comprises one or more of alcohol, volatile solvents, antihistamines, and loperamide hydrochloride.

7. The urinary catheter product of claim 1, wherein the compound comprises a thickening agent that maintains or increases the viscosity of the urethral mucus.

8. The urinary catheter product of claim 7, wherein the thickening agent comprises a gelling agent.

9. The urinary catheter product of claim 7, wherein the thickening agents comprises one or more of agar and gelatin.

10. The urinary catheter product of claim 7, wherein the thickening agent comprises a polysaccharide.
